(19) United States
(12) Patent Application Publication
(10) Pub. No.: US 2024/0157078 A1
(43) Pub. Date: May 16, 2024

(54) FUNCTIONAL POWDER DELIVERY DEVICE AND SYSTEM THEREOF

(71) Applicant: NATIONAL CHENG KUNG UNIVERSITY, Tainan City (TW)

(72) Inventors: Xi-Zhang LIN, Tainan City (TW); Ya-Wen SHIH, Tainan City (TW); Sheng-Jye HWANG, Tainan City (TW)

(21) Appl. No.: 17/988,041

(22) Filed: Nov. 16, 2022

(51) Int. Cl. A61M 16/06; A61B 17/12; A61M 16/08; A61M 16/20

(52) U.S. Cl. CPC A61M 16/0666; A61B 17/12181; A61M 16/0816; A61M 16/208; A61B 2017/12004; A61M 2202/064

(57) ABSTRACT

The present invention discloses a functional powder delivery device for precise locating and quantifying when delivering the functional powder to a site in need in a patient. Meanwhile, the device avoids negative pressure effect in the output catheter when airflow is switched, and thus prevents pollution or jam in the output catheter due to splash or back flow of body fluid. The device ensures tube clearance of the output catheter by maintaining continuous airflow, and a special design of airflow switch allows switching to airflow of large volume to carry the powder when required. Through continuous positive airflow in the tube system, the functional powder can be delivered to the site in need by a continuous powder flow, which overcomes drawbacks of the delivery devices in prior arts. In addition, the design of foot pedal can help endoscopist for a solo operation. The serial connecting parts can adapt for various size of bottles of various types of functional powder.

### FIELD OF THE INVENTION

The present invention is related to a device for airflow controlling and for delivery of functional powder to a human body; on the other hand, the device is used for delivering functional powder to a human body cavity.

### BACKGROUND OF THE INVENTION

For therapeutical or healthcare purposes, pharmaceuticals are used for inducing innate repairment of body function through oral, topical or injectable administration. Pharmaceuticals are also delivered along with invasive medical procedures towards a specific lesion, for example, to induce internal hemostasis, to close perforations in a body cavity or to target a tumor. On the other hand, when a human body lacks particular nutrients or certain synbiotics, supplements of nutrients or health food in addition to daily meals are necessary so that the human body obtains adequate nutrition or improves synbiotic flora, and acquires comprehensive healthcare effects.

In prior arts, besides aforementioned measures for delivery of drugs or nutrition, a local targeted delivery can also be performed with collocation of a conduit and an introductive device. For instance, the conduit is positioned to a lesion such as a bleeding site in a stomach, and therapeutic agents including regular therapeutic materials such as hemostatic powder or hardening agents are injected through lumen of the conduit. However, conduit-directed injection produces relatively weak injection flows. The therapeutic materials may disperse and spread thin when confronted with massive bleeding. Such drawbacks render hemostasis uncontrollable and increase risks of patients.

In addition, most of gastrointestinal probiotics might be eventually deactivated by factors including gastric acids or bile salts when delivery thereof depends on conventional measures. Therefore, mainstream intra-cavity delivery of gastrointestinal probiotics relies on capsules or microcapsules for packing probiotics so as to control the release thereof inside gastrointestinal tracts. Nonetheless, the measures as described hereinabove require a comprehensive consideration of capsule dissolution and variations of digestive ability amongst individuals. Thus, successful inoculation of the probiotics or synbiotics could not be guaranteed. Furthermore, probiotic flora varies amongst different gastrointestinal regions, which makes precise targeted delivery of probiotics a significant issue in the industry.

Currently, local delivery by a conduit is a widely-used measure. To serve purposes on local, precise and quantified delivery of therapeutical agents or healthcare products, powder is considered a most ideal delivery form. Nevertheless, conventional powder delivery device depends on air for pushing powders, and a broad spectrum of issues are yet to be addressed, such as uneven spraying caused by turbulent airflows, complicated hand maneuver, instable supply of air pressure, or negative pressure effects resulting from airflow switching, which leads to splash or back flow of body fluids or blood to jam or to contaminate the conduit. Hence, a delivery device ensuring stable air pressure, even delivery amount of powder and preventing negative pressure effects requires to be developed.

Disclosed in Pat. TWI719844B is a “POWDER SPRAY HEMOSTATIC DEVICE” which includes a medical-grade gas generator, a gas delivery controller, a powder container, and a powder delivery tube. The medical-grade gas generator comprising a main gas supply tube to output gas and connected to the gas delivery controller. The gas delivery controller comprising a first and a second gas supply tubes and controlling timing of both. The first gas supply tube is connected to the powder container, and the powder container is disposed with a one-way gas supply valve to prevent powder from flowing backward. The powder delivery tube is connected to an outlet end of the powder container, disposed with a powder passage inside, and a gas supply manifold on tube wall to communicate with the powder passage. The second gas supply tube is connected to the gas supply manifold, and the end of powder delivery tube connects to a powder spraying tube. Through continuous air supply in the second gas supply tube, the powder spray hemostatic device is able to maintain the powder spraying tube clear, but an instantaneous vacuum would be produced in the powder spraying tube when the gas supply tube stops air supply upon switch to the other gas supply tube for large air volume to push the powder. Such vacuum creates an instantaneous negative pressure and pulls back body fluids, mucous or blood, and contaminating the powder spraying tube.

Disclosed in Pat. TWI763510B is a “POWDER MEDICINE DELIVERY DEVICE” used with a medicine bottle. The medicine bottle accommodates a powder medicine and has an unfilled space not yet been filled with the powder medicine. The powder medicine delivery device includes a base body, a medicine feeding pipe, an air supply pipe, and an air supply member. The base body includes a coupling end. The medicine feeding pipe is inserted into the base body and has a medicine feeding end extending out from the coupling end; the air supply pipe is inserted into the base body and has an air supply end extending out from the coupling end, wherein the air supply end has an extension length greater than that of the medicine feeding end; the air supply member is connected to the air supply pipe. The medicine bottle is coupled to the coupling end of the base body so that the medicine feeding end and the air supply end are inserted into the medicine bottle, wherein the medicine feeding end extends into the powder medicine, and the gas supply end extends into the unfilled space. Air is introduced into the unfilled space of the medicine bottle from the air supply member so that the powder medicine is output from the medicine feeding pipe as being subjected to a pressure. The medicine bottle requires an unfilled space so as to allow air input from the air supply end to produce air pressure for pushing powder through the medicine feeding end to the medicine feeding pipe. The medicine feeding pipe extends out from the coupling end, which makes discontinuous powder flow when powder storage level is lower than height of the medicine feeding end. Precise control of powder amount could not be performed with the medicine powder delivery device, and residue as well as waste of medicine powder are inevitable.

Disclosed in Pat. TWM628518U is “POWDER PROPELLING DEVICE AND ITS AIR FLOW CONTROLLER” relating to a powder propelling device and its air flow controller. The medicine powder pushing device can be connected to an inner chamber intubation tube, and is controlled by the air flow controller to push the medicine powder in a medicine bottle into the inner chamber intubation tube. Though timing of pushing the medicine powder can be controlled by partializing air pressure with the device, it depends on only one end for airflow input to push medicine powder, and thus instantaneous negative pressure issues inside the intubation tube can not be completely prevented. Similarly, the powder propelling device could not avoid pulling back body fluid from internal cavity into the intubation tube so that the tube is polluted or jammed.

Disclosed in Pat. US20210379302A1 are “MEDICAL SCOPES FOR DELIVERING THERAPEUTIC AGENTS” suitable for delivering a therapeutic agent to a target site. In one example, the system comprises a container for holding the therapeutic agent, and a pressure source having pressurized fluid, wherein the pressure source is in selective fluid communication with at least a portion of the container. A catheter is placed in fluid communication with the container, and has a lumen sized for delivery of the therapeutic agent to a target site. A housing is configured to securely retain the container. The system further comprises a camera having a camera head coupled to the catheter, wherein the camera provides a visual image of the target site during delivery of the therapeutic agent. However, the therapeutic agent depends on the pressure source to push air inside the container so as to overcome gravitational force and enters the lumen. In initial phase of usage, stable output of the powder can be achieved. When storage level of the therapeutic agents is lower than the lumen for delivery, the floating therapeutic agents blown by the air could not ensure that powder enters the lumen in a continuous and even manner. Therefore, a continuous powder flow could not be guaranteed, which implies powder residing and quantifying issues.

As shown in FIG. 5, a commercial product from Endoclot® (Cat. K162197) is a compressing type medicine delivery device. The device is provided with a powder storage bottle having a plurality of collars on the bottle wall, making the powder storage bottle compressible. The powder storage bottle is connected to a spraying tube whose end can be introduced to a site in need through an endoscopic conduit. Medicine inside the powder storage bottle is then sprayed out by compressing the powder storage bottle. The product has simplified components and is easy to operate. However, after repetitive pressing the compressible powder storage bottle, medicine powder tends to accumulate at the collaring section, which makes it difficult to press and to produce uneven pressure, and results in inhomogeneous spraying when medicine powder is ejected. Such design renders medicine powder difficult to quantify and tend to reside in the collaring section.

### SUMMARY OF THE INVENTION

To address issues encountered in prior arts as described hereinabove, a functional powder delivery device (100) is provided in the present invention for delivery of functional powder, such as powder-formed therapeutics or health food, to a site in need in a patient. The functional powder delivery device (100) takes advantages of continuous airflow to keep the output catheter clear, and achieves at powder delivery by airflow of large volume and continuity via an air switch of a special design so that negative pressure effects caused by airflow switch can be avoided. Meanwhile, splash or back flow of body fluid, which contaminates the output catheter, is also prevented. The powder can be delivered in a continuous and even manner, and switch is fast while quantification is precise.

The functional powder delivery device (100) disclosed in the present invention comprises: a first air supply catheter (1) with one end connected to an air source (G); a second air supply catheter (2) with one end connected to the air source (G) so as to form a nasal cannula-like structure with the first air supply catheter (1); an airflow regulator (11) provided on the first air supply catheter (1) for regulating a first airflow in the first air supply catheter (1) to pass through or to drain from the first air supply catheter (1); a powder channel (3), which is a hollow body for a functional powder to pass through, comprising, sequentially, a channeling section (31), a buffering section (32) and a connecting section (33), wherein one end of the channeling section (33) away from the buffering section (32) is serial connected with a powder storage bottle (B) storing the functional powder, and cross sectional area of the buffering section (32) converges from the channeling section (31) to the connecting section (33); and an output catheter (T) serial connected to one end of the connecting section (33) away from the buffering section (32) so as to guide the functional powder to a site in need, wherein one end of the first air supply catheter (1) aerodynamically communicates with the channeling section (31) via a first opening (311) formed on one sidewall of the channeling section (31); and the other end of the second air supply catheter (2) aerodynamically communicates with the connecting section (33) via a second opening (331) formed on one sidewall of the connecting section (33), when the airflow regulator (11) keeps the first airflow passing through the first air supply catheter (1), the first airflow flows into the powder channel (3) to carry the functional powder from the powder channel (3) into the output catheter (T), wherein a second airflow in the second air supply catheter (2) continuously flows into the output catheter (T) to keep a positive pressure in the output catheter (T).

Preferably, inner side wall of the channeling section (31) and inner side wall of the buffering section (32) form, an included angle α, and the included angle α is greater than 90 degrees and less than 180 degrees.

Preferably, axis of the second air supply catheter (2) and axis of the connecting section (33) form an included angle θ neighboring to the buffering section (32), and the included angle θ is greater than 0 degree and less than or equal to 90 degrees.

Preferably, the second air supply catheter (2) is further provided with an airflow adjuster (21) for adjusting cross sectional area of the second air supply catheter (2) so as adjust flow volume of the second airflow.

Preferably, airflow volume of the first air supply catheter (1) is greater or equal to airflow volume of the second air supply catheter (2).

Preferably, a first check valve (V1) is provided between the first opening (311) and the first air supply catheter (1) to avoid the functional powder from reversibly flowing into the first air supply catheter (1).

Preferably, a second check valve (V2) is provided between the second opening (331) and the second air supply catheter (V2) to avoid the functional powder from reversibly flowing into the second air supply catheter (2).

Preferably, the airflow regulator (11) is connected to the first air supply catheter (1) through an air-draining catheter (T1).

Preferably, the airflow regulator (11) comprises: a base seat (111), a top pedal (112) and an elastomer (S), wherein one end of the top pedal (112) is pivotally connected to one end of the base seat (111), the elastomer (S) is provided between the top pedal (112) and the base seat (111), and an opened end of the air-draining catheter (T1) is installed on the base seat (111); when the top pedal (112) falls onto the base seat (111), the top pedal (112) presses the opened end of the air-draining catheter (T1) so as to close the opened end thereof, such that air in the air-draining catheter (T1) stops draining from the opened end of the air-draining catheter (T1), and the airflow directly carries the powder to the target sites.

Preferably, the powder storage bottle (B) is serial connected to the channeling section (31) further through an adaptor (R), wherein the adaptor (R) comprises: an outer periphery closely fitting the channeling section (31); and an inner periphery closely fitting bottleneck of the powder storage bottle (B), wherein the perimeter of the inner periphery optionally variates with the size of bottleneck of the powder storage bottle.

Preferably, the functional powder is manufactured with materials comprising antibiotics, bacteriostatics, hemostatics, hardening agents, analgesics, chemotherapeutic agents, biosimilars, targeted therapeutic agents, gastrointestinal probiotics or nutritional supplements selected from a group consisting of vitamins, amino acids, proteins, collagens and lipids.

In another aspect, to achieve precise orientation and delivery of functional powder, the present invention further discloses a body cavity functional powder delivery system for delivering a functional powder to a site in need inside a human body cavity, comprising: a functional powder delivery device (100) as aforementioned hereinabove; and an endoscopic device comprising: an endoscopic device comprising an endoscopic catheter, wherein the output catheter (T) wear and set in the endoscopic catheter so that a user delivers the functional powder by guiding the output catheter (T) to the site in need during therapeutic endoscopy.

### BRIEF DESCRIPTION OF THE DRAWINGS

FIGS. 1A to 1D are assembly drawings to demonstrate structure and assembly of components of the functional powder delivery device in the present invention;

FIG. 2 is an exemplary assembly drawing to illustrate the foot pedal structure of the airflow regulator; FIGS. 3A to 3B are exploded drawings to demonstrate serial connection of the powder storage bottle and the powder channel in a specific manner; FIG. 4A is a cross-sectional drawing to illustrate detailed structure of the functional powder delivery device encompassed by the outer shell; FIG. 4B presents a side view of the functional powder delivery device encompassed by the outer shell.

FIG. 5 illustrates a commercial product from Endoclot® (Cat. K162197).

### DETAILED DESCRIPTION OF THE INVENTION

Hereinafter are embodiments to exemplify and explain structures and maneuvers of each component in the present invention so as to illustrate features and spirits of the present invention, but not to limit implementation of the present invention.

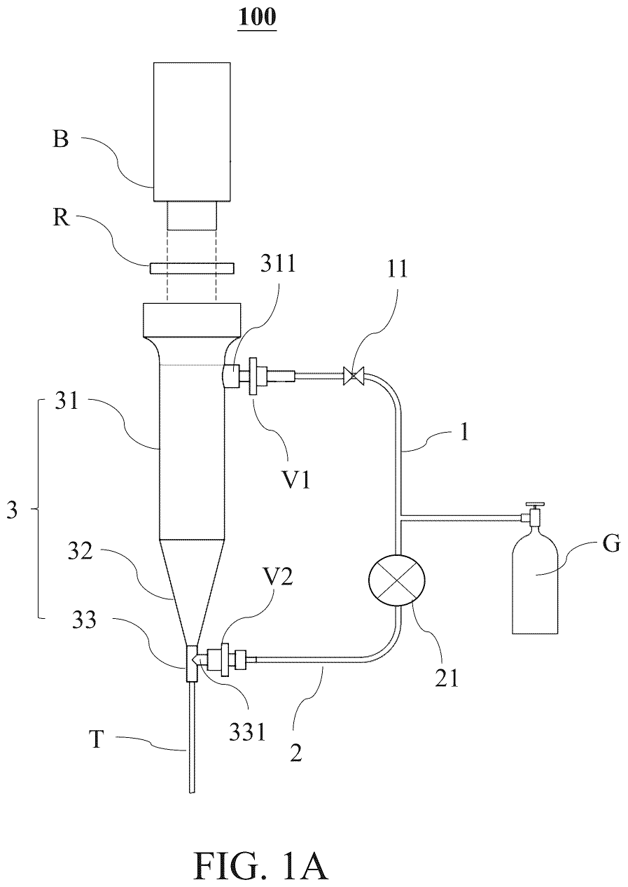

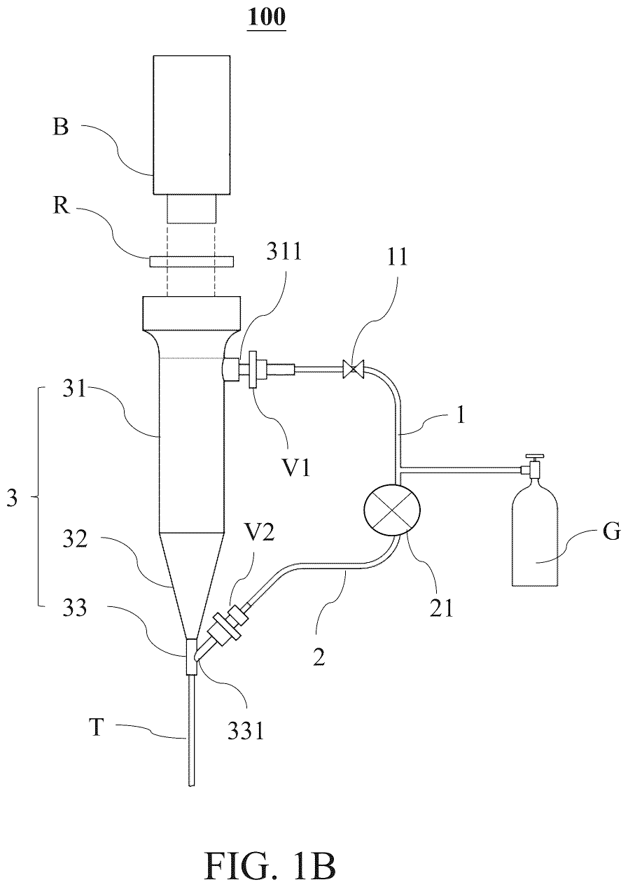

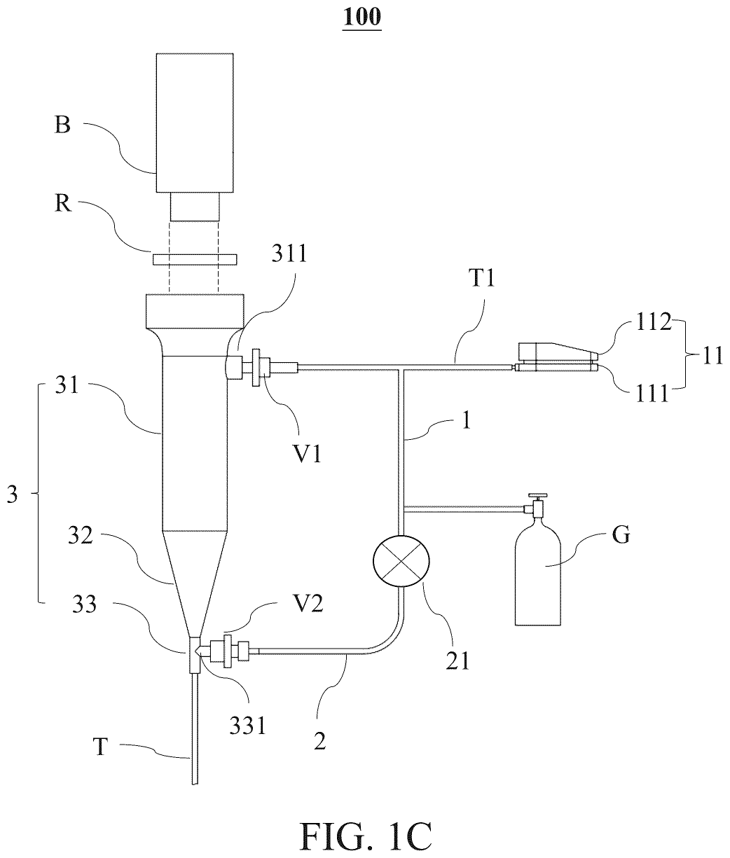

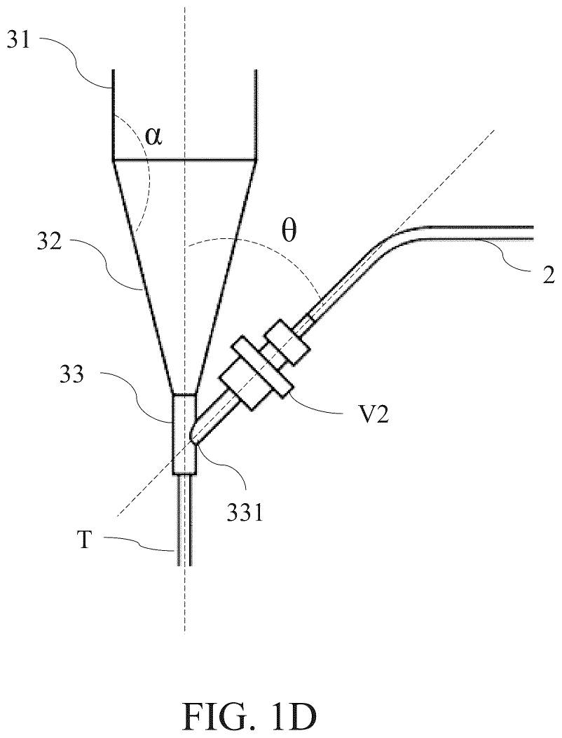

Please refer to FIGS. 1A to 1C. One aspect of the present invention is to provide a functional powder delivery device comprising: a first air supply catheter (1) with one end connected to an air source (G); a second air supply catheter (2) with one end connected to the air source (G) so as to form a nasal cannula-like structure with the first air supply catheter (1); an airflow regulator (11), e.g. a foot pedal, provided on the first air supply catheter (1) for regulating a first airflow in the first air supply catheter (1) to pass through or to drain from the first air supply catheter (1); a powder channel (3), which is a hollow body for a functional powder to pass through, comprising, sequentially, a channeling section (31), a buffering section (32) and a connecting section (33), wherein one end of the channeling section (33) away from the buffering section (32) is serial connected with a powder storage bottle (B) storing the functional powder, and cross sectional area of the buffering section (32) converges from the channeling section (31) to the connecting section (33), preferably, the buffering section (32) forms a cone-shaped structure, e.g. a Taylor cone; and an output catheter (T) serial connected to one end of the connecting section (33) away from the buffering section (32) so as to guide the functional powder to a site in need, wherein one end of the first air supply catheter (1) aerodynamically communicates with the channeling section (31) via a first opening (311) is formed on one sidewall of the channeling section (31); and the other end of the second air supply catheter (2) aerodynamically communicates with the connecting section (33) via a second opening (331) is formed on one sidewall of the connecting section (33). Specifically, when the airflow regulator (11) keeps the first airflow passing through the first air supply catheter (1), the first airflow flows into the powder channel (3) to carry the functional powder from the powder channel (3) into the output catheter (T), while a second airflow in the second air supply catheter (2) continuously flows into the output catheter (T) to assist the powder flow and to keep a positive pressure in the output catheter (T).

In various embodiments, the air source (G) is a medical grade air generator which is able to generate an air source with air pressure between 3 to 60 psi, but not limited to this. For instance, the air source (G) can be an air source mounted on a wall in hospital or a large medical institute. The air source (G) is pressurized and dried by specifically designed air compressor, and is delivered to a medical space in need, or the air source (G) can be supplied by a pressurized gas cylinder. In some exemplary embodiments, the air source (G) generates air pressure between 3 to 60 psi, preferably between 5 to 15 psi, and the airflow volume is maintained at 1 to 15 liters per minutes (L/min), preferably between 2.5 to 5.0 L/min. In one or various embodiments, the functional powder delivery device (100) delivers about 1.0 gram of the functional powder to the site in need per second.

In various embodiments, the air source (G) delivers air to the first air supply catheter (1) and the second air supply catheter (2), and the air merges into a unidirectional airflow in the powder channel (3) towards the output catheter (T). A user can regulate the flow volume of the air passing through from the first air supply catheter (1) to the powder channel (3) via the airflow regulator (11), so as to switch the airflow to continue or to stop. In practical use, the longitudinal axis of the powder channel (3) is vertical to the ground and one end of the powder channel (3) is serial connected to a powder storage bottle (B) containing a functional powder. The functional powder falls freely to the channeling section (31) by gravitational force, and piles up in the buffering section (32). When powder delivery is not conducted, the user stops the airflow in the first air supply catheter (1) via the airflow regulator (11), and the airflow is weakened. Air pressure in the channeling section (31) is far insufficient for breaking bridging effect between powder grains, which makes the powder remain piled up in the buffering section (32). When powder delivery is conducted, the user maintain the airflow unimpeded in the first air supply catheter (1) via the airflow regulator (11), and a continuous airflow forms thereafter. Being pushed by the airflow in the channeling section (31), the powder passes through the connecting section (33) from the buffering section (32) into the output catheter (T). Several manners are exemplified to stop the airflow in the first air supply catheter (1) so as to control air pressure in the channeling section (31), including: reducing the cross-sectional area of the first air supply catheter (1), or forming an air outbound hole on the first air supply catheter (1) so that the airflow leaks out of the first air supply catheter (1) and does not merge with the airflow in the second air supply catheter (2).

In various embodiments, please refer to FIG. 1D, inner side wall of the channeling section (31) and inner side wall of the buffering section (32) form an included angle α, and the included angle α is greater than 90 degrees and less than 180 degrees. It should be noted that the powder tends to pile up at the junction of the channeling section (31) and the buffering section (32) when the included angel α is closer to 90 degrees. When the air continues to pass through the channeling section (31), powder in the central portion of the channeling section (31) enters the connecting section (33), and is delivered to the site in need by the output catheter (T). As the included angle α is closer to 90 degrees, the buffering section (32) is much like a solid wall to the remaining powder. The powder does not scroll with the airflow easily because the included angle is too small, and remains piled up at the junction of the channeling section (31) and the buffering section (32), which causes rat-holing effect. Therefore, in various embodiments, the included angle ranges between 125 to 170 degrees, preferably between 160 to 170 degrees, so that the functional powder continues to scroll from the buffering section (32) to the connecting section (33) under pushing of the airflow, and rat-holing effect is overcome. Wastes of the powder due to piling up are reduced to less than 0.1% of the functional powder resides in the powder storage bottle (B)-after use.

In various embodiments, axis of the second air supply catheter (2) and axis of the connecting section (33) form an included angle θ neighboring to the buffering section (32), and the included angle θ is greater than 0 degree and less than or equal to 90 degrees, preferably between 30 to 60 degrees, more preferably between 40 to 50 degrees. In one preferred embodiment, the included angle θ is 45 degrees. With such arrangement of the catheters, tubing of the output catheter (T) can be maintained clear whenever the delivery of powder is continued or ceased. Furthermore, reverse flowing of airflow into the output catheter (T) can be avoided so that reduction of air pressure from the first air supply catheter (1) to the channeling section (31) is also prevented.

In some embodiments, volume of the first airflow is greater or equal to volume of the second airflow so that the unidirectional airflow is formed by merging the first airflow and the second airflow to ensure unidirectional delivery of the powder, and meanwhile tubing of the output catheter (T) is maintained clear by the second airflow. In some preferred embodiments, a ratio of volume of the first airflow to volume of the second airflow (2) is between 1.0:(0.01 to 1.0), preferably between 1.0:(0.1 to 1.0), and more preferably between 1.0:(0.5 to 1.0). On the other hand, in order to adjust airflow volume, as shown in FIGS. 1A to 1C, the second air supply catheter (2) is further provided with an airflow adjuster (21) for adjusting cross-sectional area of the second air supply catheter (2) so as to adjust flow volume of the unidirectional airflow in the output catheter (T), and a positive air pressure is maintained in the output catheter (T) thereafter. The airflow adjuster (21) can be exemplified by a roller-type, a valve-type or a hydraulic-type flow regulator, and not limited to this. Any type of flow regulator that allows adjustment of the passing-by airflow volume through changing cross-sectional area of the second air supply catheter (2) is included.

Preferably, to avoid the functional powder from reversibly flowing into the first air supply catheter (1) and leads to contamination or obstruction of the tubing, a first check valve (V1) is further provided between the first opening (311) and the first air supply catheter (1); on the other hand, a second check valve (V2) is optionally provided between the second opening (331) and the second air supply catheter (2) to avoid the functional powder from reversibly flowing into the second air supply catheter (2).

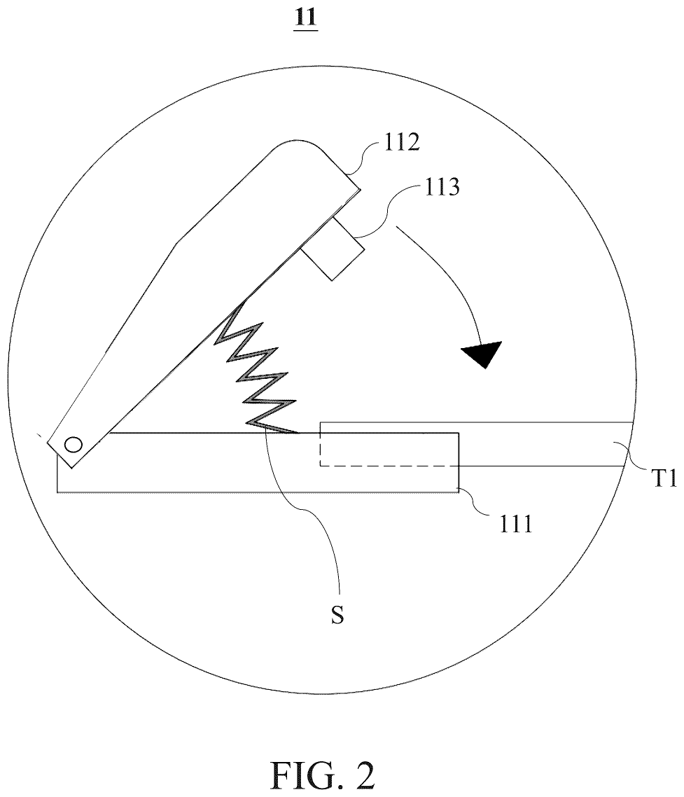

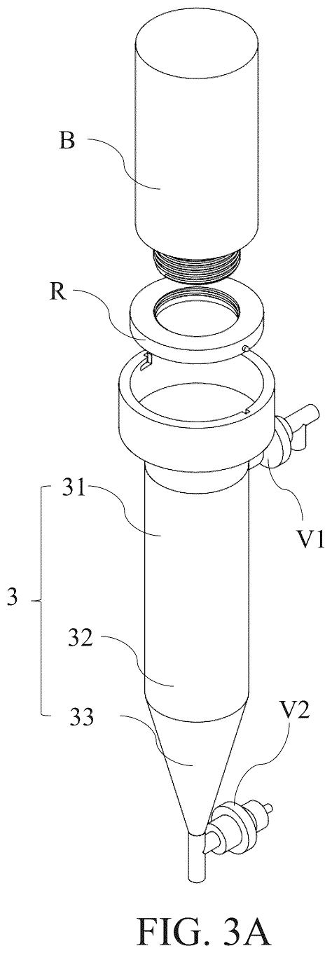

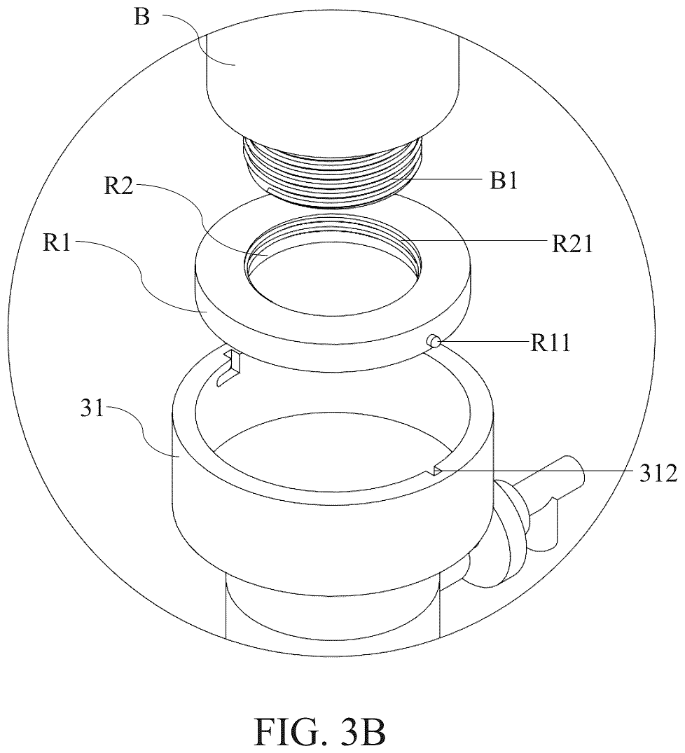

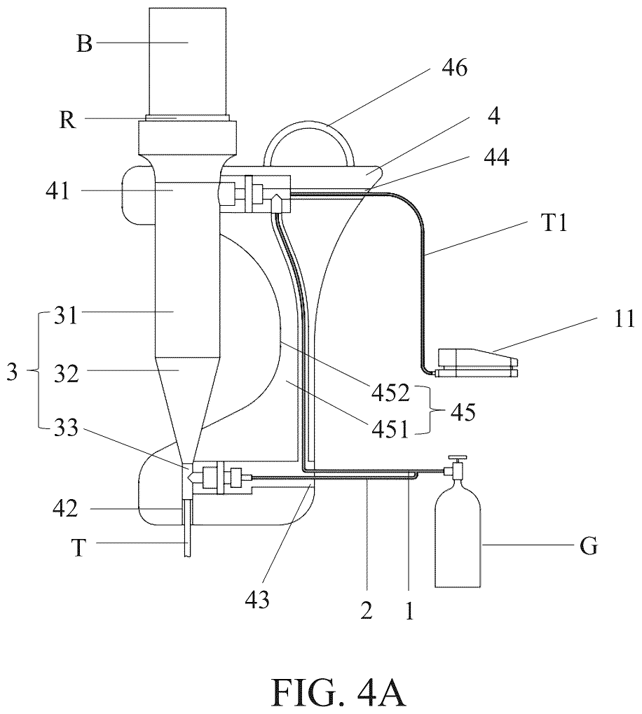

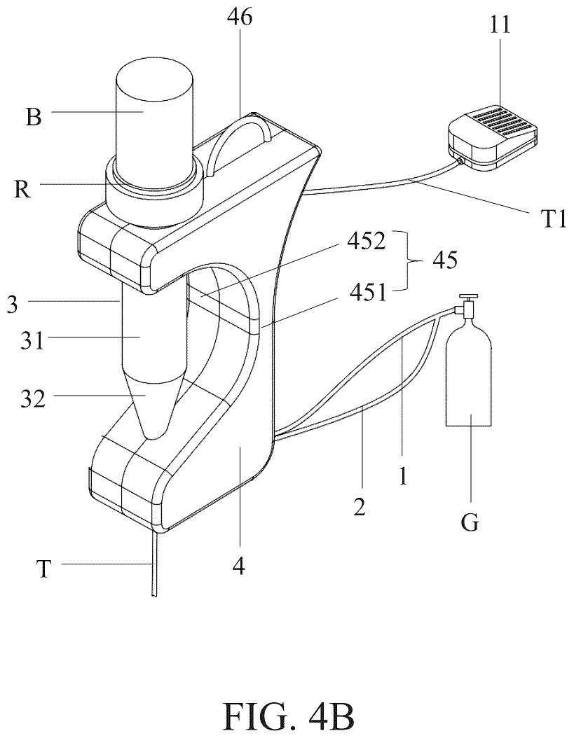

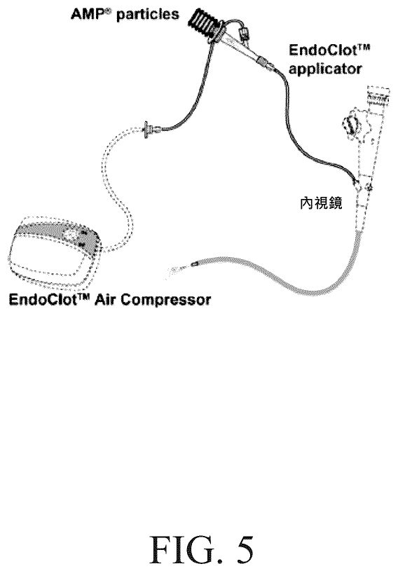

Shown in FIG. 1C is one of preferred embodiments in the present invention, wherein the airflow regulator (11) is connected to the first air supply catheter (1) through an air-draining catheter (T1) so as to realize remote control of the airflow regulator (11). More preferably, as shown in FIG. 2, the airflow regulator (11) can be a foot pedal structure so that an user can control airflow in the first air supply catheter (1) with one single foot stepping thereon when the user locates the site for powder delivery when the output catheter (T) is operated by both hands of the user. Such arrangement allows solo operation of the functional powder delivery device (100). Please continue on FIG. 2, the airflow regulator (11) comprises a base seat (111), a top pedal (112) and an elastomer (S), wherein one end of the top pedal (112) is pivotally connected to one end of the base seat (111), the elastomer (S) is provided between the top pedal (112) and the base seat (111), and an opened end of the air-draining catheter (T1) is installed on the base seat (111); when the top pedal (112) falls onto the base seat (111), the top pedal (112) presses the opened end of the air-draining catheter (T1) so as to close the opened end thereof, such that air in the air-draining catheter (T1) stops draining from the opened end of the air-draining catheter (T1), and the airflow directly carry the powder to the target sites. In some embodiments, the airflow regulator (11) further comprises a locking tooth (113) installed on the top pedal (112) in a corresponding position to an opened end of the air-draining catheter (T1). When the top pedal (112) falls onto the base seat (111), the locking tooth (113) presses tightly to close or to block the opened end. Specifically speaking, the elastomer (S) is installed for moving the top pedal (112) away from the base seat (112), and the opened end of the output catheter (T1) is relieved thereby. Airflow in the first air supply catheter (1) continuously drains out of the tubing through the air-draining catheter (T1), weakening the air pressure in powder channel (3) so that powder delivery is stopped.

Please refer to FIGS. 1A to 1C. Required amount of the functional powder varies in different situations of usage. In order to make the functional powder delivery device (100) able to be serial connected to the powder storage bottle (B) with bottlenecks of various sizes. Preferably, the powder storage bottle (B) is serial connected to the channeling section (31) further through an adaptor (R), wherein the adaptor (R) comprises an outer periphery section (R1) closely fitting the channeling section (31); and an inner periphery section (R2) closely fitting bottleneck of the powder storage bottle (B). Practically, the adaptor (R) closely fits the channeling section (31) or the powder storage bottle (B) in a variety of manners including bolt connection, tight match, interference fit, or tongue-groove structure, but not limited to this. On the other hand, the material or the surface structure of the adaptor (R) is also not limited to any specific species.

In one exemplary embodiment, the adaptor (R) can be an elastic ring made of elastic material. The outer periphery of the powder storage bottle (B) bottleneck squeezes the elastic ring against the inner side wall of the channeling section (31) so as to close fit and fix each other. In another exemplary embodiment, the adaptor (R) is connected to the channeling section (31) by an interference fit structure. Specifically, shape of the outer periphery section (R1) of the adaptor (R) is similar to the peripheral shape of channeling section (31) inner side wall. For example, when the outer periphery section (R1) is a round shape, and the peripheral shape of channeling section (31) inner side wall is also a round shape with a smaller radius than that of the outer periphery section (R1). When the adaptor (R) is placed into the channeling section (31) at one end away from the buffering section (32), the outer periphery section (R1) squeezes against the channeling section (31) inner side wall and results in slight deformation thereof. Hereby, the adaptor (R) and the channeling section (31) achieves an interference-fitting status. In one another exemplary embodiment, the adaptor (R) is connected to the channeling section (31) by a tongue-groove structure, wherein an outer groove (R11) is formed on the outer periphery section (R1) of the adaptor (R), and the outer groove (R11) snaps in an inner wall tongue (312) formed correspondingly on inner side wall of the channeling section (31). In yet another exemplary embodiment, the adaptor (R) is bolt-connected to the channeling section (31), wherein a plurality of outer threads are formed on the outer periphery section (R1) of the adaptor (R), and the outer threads correspond to inner wall threads formed on inner side wall of the channeling section (31). In the aforementioned exemplary embodiments, the powder storage bottle (B) can also be connected to the adaptor (R) through bolt connection, tight match, interference fit, or tongue and groove joint so as to be serial connected to the channeling section (31).

In some preferred embodiments, as shown in FIGS. 3A to 3B, the adaptor comprises a plurality of inner threads (R21) formed in the inner periphery section (R2), wherein the inner threads (R21) is positioned correspondingly to bottleneck threads (B1) formed on outer periphery of bottleneck of the powder storage bottle (B); in addition, the outer periphery section (R1) of the adaptor are set with an outer groove (R11), wherein the outer groove (R11) is formed correspondingly to inner wall tongue (312) formed in the inner sidewall periphery of the channeling section (31). In this embodiment, the outer groove (R11) is inserted into the inner wall tongue (312), wherein the outer groove (R11) and the inner wall tongue (312) move relatively so as to fasten each other, and the powder storage bottle (B) is serial connected to the powder channel (3). Herein only a few exemplary embodiments are described so as to illustrate tightly serial connection of the powder storage bottle (B) to the powder channel (3) via the adaptor (R). In the present invention, users can replace adaptors (R) of various sizes corresponding to powder storage bottles (B) of various volumes or bottleneck sizes so as for application of the functional powder delivery device (100) on a diversity of medical scenes. When species or amounts of the functional powder variate under various circumstances, the user can freely connect the powder storage bottle (B) to the powder channel (3) serially so as to administrate functional powder that is required, without being limited to a particular size of bottleneck of the powder storage bottle (B).

In some preferred embodiments, the functional powder delivery powder (100) is further encompassed by an outer shell (4) as shown in FIGS. 4A to 4B. The outer shell (4) is formed with a first passthrough (41) and a second passthrough (42), wherein the channeling section (31) is worn in the first passthrough (41), and the connecting section (33) is worn in the second passthrough (42) so that the powder channel (3) is fixed on the outer shell. The outer shell (4) is formed with an air supply pore (43) so as to wear in the first air supply catheter (1) and the second air supply catheter (2), and the outer shell (4) is formed with an airflow controlling pore (44) so as for installation of the airflow regulator (11). Preferably, the air-draining catheter (T1) is worn in the airflow controlling pore (44) to connect the airflow regulator (11) to the first air supply catheter (1).

In preferred embodiments, the outer shell (4) further comprises a holding section (45) set between the first passthrough (41) and the second passthrough (42). As illustrated in FIGS. 4A to 4B, the holding section (45) comprises a concave section (451) formed on one side of the outer shell (4) neighboring to the powder channel (3). The concave section (451) and the powder channel (3) together determines a holding space (452) so as to allow fingers of a user to pass through and hold the functional powder delivery device (100). More preferably, the outer shell (4) is set with a hanging section (46) so that the functional powder delivery device (100) can be hung on a drip rack, a wall hook or anywhere there is a hook structure, and not limited to this.

In another aspect, the present invention further provides a body cavity functional powder delivery system for delivering a functional powder to a site in need inside a human body cavity, comprising a functional powder delivery device (100) as aforementioned hereinabove; and an endoscopic device comprising an endoscopic catheter, wherein the output catheter (T) wear and set in the endoscopic working channel; a camera coupling to a frontal end of the endoscopic catheter for acquiring an image of the site in need; and a displayer signally connected to the camera for displaying the image, wherein a user delivers the functional powder by guiding the output catheter (T) to the site in need based on the image during therapeutic endoscopy.

In practical use, a user can hang the functional powder delivery device (100) perpendicular to the ground via the hanging section (46), and turns on the air source (G) so as to generate a continuous airflow. The airflow regulator (11) is maintained open so that airflow in the first air supply catheter (1) leaks out of the catheter and the functional powder remains piled up in the powder channel (3). Subsequently, the user operates the endoscopic device to insert the endoscopic catheter into a patient's body cavity, and locates distal end of the endoscopic catheter via the displayer demonstrating images acquired by the camera. For example, the working area is a wound with massive bleeding inside the body cavity, and the user can step on and close the airflow regulator (11) so that airflow passes through the first air supply catheter (1), entering the powder channel (3) and forming a continuous airflow to push the functional powder into the output catheter (T). Eventually, the functional powder is delivered to the wound precisely. In this exemplary embodiment, the functional powder is a hemostatic powder which promotes hemostasis to close the bleeding site.

In the present invention, the functional powder means to be any powder or particles manufactured with materials having medical or healthcare effects. For example, the material can be antibiotics, bacteriostatics, hemostatics, hardening agents, analgesics, chemotherapeutic agents, biosimilars, targeted therapeutic agents or any pharmaceuticals absorbable to mucosa, but not limited to this. The material can also be exemplified by lyophilized gastrointestinal probiotics or nutritional supplements such as vitamins, amino acids, proteins, collagens or lipids, but not limited to this. In addition, the functional powder delivery device (100) provided in the present invention is not limited to application inside a body cavity, but can also serves therapeutic or healthcare purposes by delivering functional powder to a body surface.

Advantages and effects of the present invention are described hereinafter:

1. The functional powder delivery device provided in the present invention solves negative pressure effects caused by discontinuity of airflow in prior arts. As unidirectional airflows are supplied from two ends of the powder channel, one of the airflow can maintain the output catheter clear when the other airflow is cut off. Such design prevents the output catheter from being polluted or obstructed by body fluids or blood which is drawn back by negative pressure.

2. The functional powder delivery device in the present invention can be connected to air source installed on a wall in a hospital, so the device is not limited by volumes of gas cylinders in prior arts. Besides, the powder is not only delivered by continuous airflow in the device, but also the airflow is unidirectional with gravitational force, making powder delivery smoother. Moreover, switch of powder delivery can be controlled instantly by simply controlling airflow, and locations as well as amount of powder delivery is much more precise.

3. The functional powder delivery device provided in the present invention overcomes rat-holing effect as implied in prior arts by design of the buffering section. A wide included angle between the buffering section and the channeling section makes the powder easier to scroll into the output catheter under collective effects of gravitational force and airflow. Residual powder in the powder channel is largely reduced, and at most the residual powder can be reduced to below 0.1%.

4. The functional powder delivery device provided in the present invention can be connected to powder storage bottles of various sizes via design of the adaptor. Users can replace the powder storage bottle according to usage scenarios so as to meet required amount of powder instantly.

5. The functional powder delivery device provided in the present invention can be operated along with an endoscopic system. Through attaching airflow regulator to output catheter, remote airflow switch is realized. The user can deliver the functional powder and simultaneously operate other devices with both hands, such as an endoscope, wherein the user simply steps on the airflow regulator by one footstep.

What is claimed is:

1. A functional powder delivery device comprising:
a first air supply catheter with one end connected to an air source;
a second air supply catheter with one end connected to the air source so as to form a nasal cannula-like structure with the first air supply catheter;
an airflow regulator provided on the first air supply catheter for regulating a first airflow in the first air supply catheter to pass through or to drain from the first air supply catheter;
a powder channel, which is a hollow body for a functional powder to pass through, comprising, sequentially, a channeling section, a buffering section and a connecting section, wherein:
one end of the channeling section away from the buffering section is serial connected with a powder storage bottle storing the functional powder, and cross sectional area of the buffering section converges from the channeling section to the connecting section; and
an output catheter serial connected to one end of the connecting section away from the buffering section so as to guide the functional powder to a site in need, wherein:
the other end of the first air supply catheter aerodynamically communicates with the channeling section via a first opening formed on one sidewall of the channeling section; and
the other end of the second air supply catheter aerodynamically communicates with the connecting section via a second opening formed on one sidewall of the connecting section, when the airflow regulator keeps the first airflow passing through the first air supply catheter, the first airflow flows into the powder channel to carry the functional powder from the powder channel into the output catheter, wherein a second airflow in the second air supply catheter flows into the output catheter to assist the powder flow and to keep a positive pressure in the output catheter.

2. The functional powder delivery device as claimed in claim 1, wherein inner side wall of the channeling section and inner side wall of the buffering section form an included angle α, and the included angle α is greater than 90 degrees and less than 180 degrees.

3. The functional powder delivery device as claimed in claim 1, wherein axis of the second air supply catheter and axis of the connecting section form an included angle θ, and the included angle θ is greater than 0 degree and less than or equal to 90 degrees.

4. The functional powder delivery device as claimed in claim 1, wherein the second air supply catheter is further provided with an airflow adjuster for adjusting cross sectional area of the second air supply catheter so as to adjust flow volume of the second airflow.

5. The functional powder delivery device as claimed in claim 4, wherein volume of the first airflow is greater or equal to the volume of the second airflow.

6. The functional powder delivery device as claimed in claim 1, wherein a first check valve is provided between the first opening and the first air supply catheter to avoid the functional powder from reversibly flowing into the first air supply catheter.

7. The functional powder delivery device as claimed in claim 6, wherein a second check valve is provided between the second opening and the second air supply catheter to avoid the functional powder from reversibly flowing into the second air supply catheter.

8. The functional powder delivery device as claimed in claim 1, wherein the airflow regulator is connected to the first air supply catheter through an air-draining catheter, and wherein the airflow regulator comprises:
a base seat, a top pedal and an elastomer, wherein one end of the top pedal is pivotally connected to one end of the base seat, the elastomer is provided between the top pedal and the base seat, and an opened end of the air-draining catheter is installed on the base seat; when the top pedal falls onto the base seat, the top pedal presses the opened end of the air-draining catheter so as to close the opened end thereof, such that air in the air-draining catheter stops draining from the opened end of the air-draining catheter, and the airflow directly carries the powder to the target sites.

9. The functional powder delivery device as claimed in claim 1, wherein the powder storage bottle is serial connected to the channeling section further through an adaptor, wherein the adaptor comprises:
an outer periphery closely fitting the channeling section; and
an inner periphery closely fitting bottleneck of the powder storage bottle, wherein the perimeter of the inner periphery optionally variates with the size of bottleneck of the powder storage bottle.

10. The functional powder delivery device as claimed in claim 1, further encompassed by an outer shell formed with a first passthrough and a second passthrough, wherein the channeling section is worn in the first passthrough, and the connecting section is worn in the second passthrough so that the powder channel is fixed on the outer shell, wherein the outer shell further comprises a holding section set between the first passthrough and the second passthrough, wherein the holding section comprises a concave section formed on one side of the outer shell neighboring to the powder channel, and the concave section and the powder channel together determines a holding space so as to allow fingers of users to pass through and hold the functional powder delivery device.

11. The functional powder delivery device as claimed in claim 1, wherein the functional powder is manufactured with materials comprising antibiotics, bacteriostatics, hemostatics, hardening agents, analgesics, chemotherapeutic agents, biosimilars, targeted therapeutic agents, gastrointestinal probiotics or nutritional supplements selected from a group consisting of vitamins, amino acids, proteins, collagens and lipids.

12. A body cavity functional powder delivery system for delivering a functional powder to site in need inside a human body cavity, comprising:
a functional powder delivery device as claimed in claim 1; and
an endoscopic device comprising an endoscopic catheter, wherein the output catheter wear and set in the endoscopic working channel so that a user delivers the functional powder by guiding the output catheter to the site in need during therapeutic endoscopy.

13. The body cavity functional powder delivery system as claimed in claim 13, wherein inner side wall of the channeling section and inner side wall of the buffering section form an included angle α, and the included angle α is greater than 90 degrees and less than 180 degrees.

14. The body cavity functional powder delivery system as claimed in claim 13, wherein axis of the second air supply catheter and axis of the connecting section form an included angle θ, and the included angle θ is greater than 0 degree and less than or equal to 90 degrees.

15. The body cavity functional powder delivery system as claimed in claim 12, wherein the second air supply catheter is further provided with an airflow adjuster for adjusting cross sectional area of the second air supply catheter so as to adjust flow volume of the second airflow.

16. The body cavity functional powder delivery system as claimed in claim 15, wherein volume of the first airflow is greater or equal to the volume of the second airflow.

17. The body cavity functional powder delivery system as claimed in claim 12, wherein a first check valve is provided between the first opening and the first air supply catheter to avoid the functional powder from reversibly flowing into the first air supply catheter.

18. The body cavity functional powder delivery system as claimed in claim 17, wherein a second check valve is provided between the second opening and the second air supply catheter to avoid the functional powder from reversibly flowing into the second air supply catheter.

19. The body cavity functional powder delivery system as claimed in claim 12, wherein the airflow regulator is connected to the first air supply catheter further through an air-draining catheter having one end connected to the first air supply catheter, and wherein the airflow regulator comprises:
a base seat, a top pedal and an elastomer, wherein one end of the top pedal is pivotally connected to one end of the base seat, the elastomer is provided between the top pedal and the base seat, and an opened end of the air-draining catheter is installed on the base seat; when the top pedal falls onto the base seat, the top pedal presses the opened end of the air-draining catheter so as to close the opened end thereof, such that air in the air-draining catheter stops draining from the opened end of the air-draining catheter, and the airflow directly to carry the powder to the target sites.

20. The body cavity functional powder delivery system as claimed in claim 12, wherein the powder storage bottle is serial connected to the channeling section further through an adaptor, wherein the adaptor comprises:
an outer periphery closely fitting the channeling section; and
an inner periphery closely fitting bottleneck of the powder storage bottle, wherein perimeter of the inner periphery optionally variates with size of bottleneck of the powder storage bottle.
